(19) United States
(12) Patent Application Publication
(10) Pub. No.: US 2007/0100162 A1
(43) Pub. Date: May 3, 2007

(54) Process for the liquefaction of lignocellulosic material

(76) Inventors: Leonardus Petrus, Amsterdam (NL); Angelika Voss, Amsterdam (NL)

Correspondence Address: SHELL OIL COMPANY, P O BOX 2463, HOUSTON, TX, 772522463, US

(21) Appl. No.: 10/582,888

(22) PCT Filed: Dec. 14, 2004
(86) PCT No.: PCT/EP04/53450

§ 371 Date: Jun. 14, 2006

(30) Foreign Application Priority Data
Dec. 15, 2003 (EP) 03257858.5

(51) Int. Cl. C07C 51/00

(52) U.S. Cl. 562/515

(57) ABSTRACT

A process for the liquefaction of lignocellulosic or cellulosic material, wherein solid lignocellulosic or cellulosic material is heated at a temperature in the range of from 100 to 300° C. in the presence of an acid catalyst and a solvent, wherein the solvent-to-solid material weight ratio is at most 50, the acid catalyst is present in a concentration of at most 50% by weight of acid based on the weight of solvent and acid, and the solvent comprises a compound having a gamma lactone group of the general molecular formula (1) wherein R₁ to R₆ each represent, independently, a hydrogen atom or an organic group connected with a carbon atom to the lactone group or the solvent comprises furfural, levulinic acid or a compound obtainable from furfural or levulinic acid by hydrogenation, dehydration, aldolcondensation, dimerisation or oligomerisation, esterification with an alcohol, or a combination of two or more of these reactions.

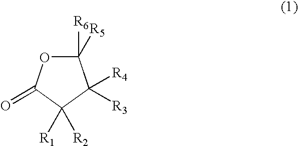

### FIELD OF THE INVENTION

The present invention relates to a process for the liquefaction of lignocellulosic or cellulosic material.

### BACKGROUND OF THE INVENTION

It is known to produce levulinic acid by controlled acid hydrolysis of lignocellulosic material. Levulinic acid is a useful compound, since its reactive nature makes it a suitable intermediate for the production of commercially interesting compounds. Reference is made to R. H. Leonard, “Levulinic Acid as Basic Chemical Raw Material”, Ind. Eng. Chem., 48 (1956) 1331-1341, in this respect.

In U.S. Pat. No. 5,608,105 for example, a process is described wherein levulinic acid is produced from cellulose or ligno-cellulose material by hydrolysing the material at a temperature between 210 and 230° C. in the presence of 1-5% weight of mineral acid in a first reactor to obtain a hydroxymethylfurfural-containing intermediate product and further hydrolysing the intermediate product at a temperature between 195 and 215° C. in the presence of 3-7.5% weight of mineral acid in a second reactor. The temperature in the first and the second reaction vessel is controlled by injection of high pressure steam. The process pressure is above 200 psig (14 bar g) in the second vessel and much higher in the first vessel.

In U.S. Pat. No. 6,054,611, a process is described wherein biomass containing cellulose and hemicellulose is mixed with sulphuric acid to form an aqueous reaction solution having about 20-40% by weight of biomass and 10-30% by weight of acid. The reaction solution is first maintained below 60° C. to decrystallise the biomass and then heated to 80-200° C. to hydrolyse the biomass to form sugars, which then react to form levulinic acid.

In the above-described prior art processes, solid biomass or lignocellulosic material is used as starting material for the production of levulinic acid. Either high pressure or a high concentration of acid is needed to be able to convert the solid material. It would be advantageous to use liquefied lignocellulosic or cellulosic material as starting material for levulinic acid production processes or other chemical processes for the conversion of lignocellulose. A liquefied starting material for levulinic acid production or other biomass conversion processes might allow milder process conditions and allows the use of heterogeneous catalysis for further processing. It would further allow separation of contaminants from the desired components in the starting material.

Reference herein to liquefied (ligno)cellulosic materials is to (ligno)cellulosic material dissolved in a liquid medium. Liquefaction of solid (ligno)cellulosic material is typically achieved by mildly heating the (ligno)cellulose with an organic solvent in the presence of an homogeneous acid catalyst. This treatment will result in some cleavage of covalent linkages of cellulose, hemicellulose and lignin and of covalent linkages between lignin and hemicellulose. Acids such as formic, acetic and propionic acid, ketones, aldehydes, sugars and lignin degradation products might be formed.

It is known that biomass or lignocellulosic material can be liquefied at ambient pressure and moderate temperature in the presence of an acid catalyst by using solvents.

In EP 472 474 A, for example, is described a process for liquefying lignocellulose substance by heating the substance at a temperature of 100 to 200° C. and ambient pressure in the presence of an acid catalyst and one or more polyhydric alcohols. Polyethylene glycol, polypropylene glycol, glycerin, ethylene glycol, 1,4-butanol, 1,6-hexanol and polycaprolactone are mentioned as possible polyhydric alcohols.

In Bioresource Technology 70 (1999) 61-67, the use of cyclic carbonates such as ethylene carbonate and propylene carbonate as solvents for the liquefaction of lignocellulosic material in the presence of an acid catalyst at 120-150° C. has been described.

### SUMMARY OF THE INVENTION

It has now been found that it is possible to liquefy lignocellulosic or cellulosic material at mild conditions by using as solvent compounds that can be derived from (ligno)cellulosic material, in particular compounds derived from acid hydrolysis processes that result in the formation of levulinic acid and, in the case that C₅-containing hemicelluloses are present in the biomass, also furfural.

Compounds having a gamma lactone group have been found to be particularly suitable as solvent for biomass liquefaction at mild conditions. Such compounds with a gamma lactone group are typically obtainable from levulinic acid, for example by hydrogenation and dehydration (in this case internal ester formation to form a lactone) or by aldolcondensation and dehydration, optionally in combination with hydrogenation. Compounds with a gamma lactone group that are not typically obtainable from levulinic acid, e.g. gamma butyrolactone also known as dihydrofuran-2(3H)-one, are also suitable as solvents for liquefaction of (ligno)cellulosic material at mild conditions.

Furfural or levulinic acid themselves or compounds obtainable from them, other than the above-defined compounds with a gamma lactone group, have also found to be suitable as solvents for liquefaction of (ligno)cellulosic material at mild conditions.

Accordingly, the present invention relates toga process for the liquefaction of lignocellulosic or cellulosic material, wherein solid lignocellulosic or cellulosic material is heated at a temperature in the range of from 100 to 300° C. in the presence of an acid catalyst and a solvent, wherein the solvent-to-solid material weight ratio is at most 50, the acid catalyst is present in a concentration of at most 50% by weight of acid based on the weight of solvent and acid, and the solvent comprises a compound having a gamma lactone group of the general molecular formula 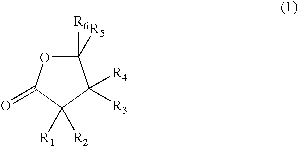
wherein R₁ to R₆ each represent, independently, a hydrogen atom or an organic group connected with a carbon atom to the lactone group.

The invention further relates to the herein-above defined process for the liquefaction of lignocellulosic or cellulosic material, wherein the solvent comprises furfural, levulinic acid or a compound obtainable from furfural or levulinic acid by hydrogenation, dehydration, aldolcondensation, dimerisation or oligomerisation, esterification with an alcohol, or a combination of two or more of these reactions.

An advantage of the process according to the present invention is that the solvent can be obtained from the (ligno)cellulosic material by either a parallel or a (ligno)cellulose conversion process step.

### DETAILED DESCRIPTION OF THE INVENTION

In one aspect of the liquefaction process according to the invention, the solvent comprises a compound with a gamma lactone group. Alternatively, the solvent comprises furfural, levulinic acid, a compound obtainable from furfural, or a compound without a gamma lactone group obtainable from levulinic acid. The solvent may comprise two or more of these compounds. Preferably, the solvent essentially consists of one or more of these compounds, i.e. compounds with a gamma lactone group, furfural, levulinic acid, compounds obtainable from furfural, or compounds without a gamma lactone group obtainable from levulinic acid, without substantial amounts of other components.

Reference herein to a compounds having a gamma lactone group is to a compound according to general molecular formula (1), wherein R₁ to R₆ are a H atom or an organic group as hereinabove defined. The total number of carbon atoms of the compound is preferably at most 20, more preferably at most 15.

Preferably, each of R₁ to R₄ is a H atom. More preferably, each of R₁ to R₄ is a H atom and R₅ is a methyl group. Particularly preferred examples of such compounds are gamma valerolactone (R₆ is a H atom), also known as 5-methyldihydrofuran-2(3H)-one, 2-methyl-5-oxotetrahydrofuran-2-carboxylic acid (R₆ is a carboxyl group), a compound with a molecular structure according to any one of molecular formulas (2) to (5), or an ester of a compound according to formula (2) or (3): 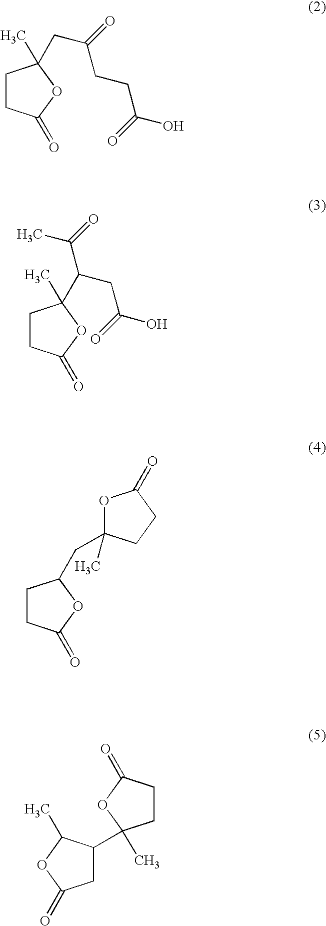
Gamma valerolactone can be obtained from levulinic acid by hydrogenation followed by dehydration (internal ester formation). The compounds according to formulas (2) to (5) are levulinic acid dimers that may be obtained by contacting levulinic acid in the presence of hydrogen with a strongly acidic catalyst having a hydrogenating function, e.g. Pd/cation-exchange resin, at elevated temperature and preferably at elevated pressure. Typical process temperatures and pressures are in the range of from 60 to 170° C. and of from 1 to 200 bar (absolute), respectively. Such process for levulinic acid dimerisation is described in detail in co-pending patent application EP 04106107.8. The catalyst and process conditions of this process are similar to those applied in the known single-step process for the production of methyl isobutyl ketone from acetone.

If in the process according to the invention the solvent is a strong acid, such as for example 2-methyl-5-oxotetrahydrofuran-2-carboxylic acid, the use of additional acid may be redundant. It is therefore possible that the solvent and the acid are the same compound.

Other compounds with a gamma lactone group suitable to be used as solvent in the liquefaction process according to the invention, which are obtainable by the above-mentioned levulinic acid dimerisation process, are the compounds with a molecular structure according to formula (6) or (7) or their esters:

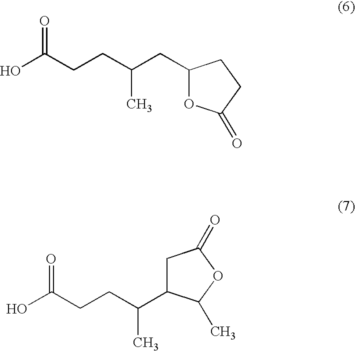

If the compound with a gamma lactone group is an ester of an acid according to molecular formula (2), (3), (6) or (7), then the ester preferably is an alkyl ester with an alcohol fragment with at most 10 carbon atoms, more preferably a linear alkyl ester with an alcohol fragment with at most 5 carbon atoms, even more preferably a methyl or an ethyl ester.

Compounds with a gamma lactone group suitable to be used as solvent in the liquefaction process according to the invention may also be formed by dimerisation or oligomerisation of alpha angelica lactone. Alpha angelica lactone may be obtained from levulinic acid by internal ester formation (dehydration) of the enol form of the carbonyl group in levulinic acid.

The compounds with a gamma lactone group are preferably obtainable from levulinic acid by hydrogenation, dehydration, aldolcondensation, dimerisation or oligomerisation, esterification with an alcohol, or a combination of two or more thereof. Examples of such compounds have been mentioned hereinabove.

Also levulinic acid, furfural or compounds without a gamma lactone group that are obtainable from levulinic acid or furfural may be used as solvent in the process according to the invention.

Examples of compounds suitable to be used as solvent in the process according to the invention that can be obtained from furfural are tetra hydrofurfuryl alcohol, furfuryl alcohol, diether of furfuryl alcohol, dimers or oligomers of furfural or furfuryl alcohol.

Examples of suitable compounds without a gamma lactone group obtainable from levulinic acid are C₅ compounds that can be obtained from levulinic acid by dehydration or dehydration in combination with hydrogenation, such as alpha-angelicalactone, 1,4-pentanediol, 1-pentanol, 2-pentanol, or methyl tetrahydrofuran. The conversion of levulinic acid in these compounds is described in more detail in U.S. Pat. No. 5,883,266.

Another example of a suitable compound obtainable from levulinic acid is 4-methyl-6-oxononanedioic acid or a mono- or di-ester thereof. Such mono- or di-ester preferably has an alcohol fragment with at most 10 carbon atoms, more preferably a linear alcohol fragment with at most 5 carbon atoms, even more preferably is the di-methyl or the di-ethyl ester. 4-methyl-6-oxononanedioic acid is the main dimer that is obtained in the above-described levulinic acid dimerisation process in the presence of hydrogen and a strongly acidic hydrogenation catalyst.

Examples of other compounds obtainable from levulinic acid that are suitable to be used as solvent are esters of levulinic acid, for example the ethylene glycol ester or alkyl esters of levulinic acid, more preferably alkyl esters with at most 10 carbon atoms in the alcohol fragment, even more preferably linear alkyl esters with at most 5 carbon atoms in the alcohol fragment, such as for example ethyl levulinate, butyl levulinate, or pentyl levulinate.

In case oligomers are used as solvent or solvent component, the oligomer will typically contain at most ten monomer units, preferably at most five, since the solvent has to be a liquid under the process conditions.

The solvent and the solid (ligno)cellulosic material are mixed in a solvent-to-solid weight ratio of at most 50. Reference herein to the weight of the solid material is to the dry solid material. Since the amount of solvent should be sufficient to wet the solid material, the solvent-to-solid weight ratio will typically be above 3.

Acid is added in such amount that the acid concentration is at most 50% by weight based on the weight of solvent and acid. The acid may be an homogeneous or a finely dispersed colloidal acid, preferably a homogeneous acid. Preferably, a strong mineral or organic acid is used. Reference herein to a strong acid is to an acid having a pKa value below 4.7. More preferably, the acid has a pKa value below 3.5, even more preferably below 2.5.

Examples of suitable strong mineral acids are sulphuric acid, para toluene sulphonic acid, phenol sulphonic acid, phosphoric acid, or hydrochloric acid. Preferred mineral acids are sulphuric acid or phosphoric acid, since they have a relatively high boiling point and thus remain liquid and stable at the process temperature at ambient pressure. Particularly preferred is phosphoric acid. Compared to sulphur-containing acids, phosphoric acid has the advantage that recovered phosphoric acid can be converted into valuable products such as fertilizers and that it is less prone to coking.

Suitable organic acids are strong organic acids that are liquid under the process conditions applied. Examples of suitable organic acids are oxalic acid, formic acid, 2-oxo-propanoic acid (pyruvic acid), lactic acid, citric acid, (1E)-prop-1-ene-1,2,3-tricarboxylic acid, malonic acid, maleic acid, glycolic acid, 2-furoic acid, 5-(hydroxymethyl)-2-furoic acid, furan-2,5-dicarboxylic acid, salicylic acid, 2,4,5-trihydroxybenzoic acid, 2,3-dihydroxysuccinic acid (tartaric acid), 2-methylenesuccinic acid (itaconic acid). Preferred organic acids are oxalic acid, 2-oxo-propanoic acid, maleic acid, (1E)-prop-1-ene-1,2,3-tricarboxylic acid, 2,3-dihydroxysuccinic acid or furan-2,5-dicarboxylic acid.

A mixture of different acids may be used.

The acid concentration is preferably at most 20% by weight, more preferably in the range of from 0.5 to 10% by weight, even more preferably of from 1 to 5% by weight. The solvent-to-solid weight ratio is preferably in the range of from 3 to 20, more preferably of from 5 to 15. The mixture of solids, solvent and acid is thus a non-aqueous mixture that may contain a small amount of water that is brought in with the acid.

In order to facilitate liquefaction, the solid (ligno)cellulose material will typically be finely divided, for example in the form of powder (e.g. sawdust), wood chips, cutted stalks, fibres or paper or the like. It will be appreciated that a smaller size of the material will result in a reduced liquefaction time. A larger size of the material may be combined with some mechanical action during the liquefaction process in order to achieve liquefaction within an acceptable time.

The mixture of solids, solvent and acid is heated at a temperature in the range of from 100 to 300° C., preferably of from 125 to 250° C., more preferably of from 150 to 210° C. It is an advantage of the process according to the invention that no high pressures are needed to achieve sufficient liquefaction of the solid material at an acceptable speed. The pressure is preferably in the range of from 0.1 to 15 bar (absolute), more preferably of from 0.5 to 10 bar (absolute), even more preferably of from 0.8 to 3 bar (absolute). Ambient pressure is most preferred. With the liquefaction process according to the invention, a considerable amount of the solid is liquefied, typically at least 50% (w/w). It is possible to liquefy more than 80% (w/w) of the solid material at ambient pressure within a few hours.

The solvent used in the liquefaction process according to the invention may be obtained from furfural or levulinic acid that is obtained from acid hydrolysis of (ligno)cellulosic material. The solvent may be obtained from the same (ligno)cellulosic material as used in the liquefaction process, either in a parallel process or in a process in series. A parallel process means that part of the (ligno)cellulosic material is subjected to an acid hydrolysis process to obtain levulinic acid, optionally in combination with furfural. The levulinic acid or furfural may then be used as such as solvent or may be further processed to obtain a solvent. The other part of the (ligno)cellulosic material is liquefied according to the process of the invention, using the solvent obtained from the same (ligno)cellulosic material. A process in series means that (ligno)cellulosic material is first liquefied according to the process of the invention and the liquefied (ligno)cellulosic material or the residue is then subjected to an acid hydrolysis process to obtain levulinic acid, optionally in combination with furfural. Part of the thus-obtained levulinic acid or furfural or a derivative thereof is then recycled to the liquefaction process as (make-up) solvent.

The acid hydrolysis process to obtain levulinic acid may for example be a process as disclosed in U.S. Pat. No. 5,608,105 or U.S. Pat. No. 6,054,611.

An advantage of the use of solvents that are derived from subsequent acid hydrolysis of the liquefied (ligno)cellulosic material is that the solvent does not need to be separated from the liquefied (ligno)cellulosic material, since the compound does not negatively influence the subsequent process step. Another advantage is that the acid catalyst does not need to be removed from the liquefied material, since the subsequent process is also a process using an acid catalyst.

### EXAMPLES

The invention will be further illustrated by means of the following non-limiting example.

### Examples 1-3

2 grams of dried birch (Betula ssp.) sawdust and 20 grams of gamma-valerolactone (γVL) and an amount of phosphoric acid (85% by weight) were loaded in a 30 ml glass flask. The flask was placed in a silicon oil bath and heated. The amount of phosphoric acid, temperature, and duration of the experiment were varied. The flasks were cooled with water to end the experiment. The percentage of residue was determined by vacuum-filtering the content of the flask, followed by washing the residue with 150 ml of acetone/water (9:1 v/v). The resulting residue was dried at 105° C. for 8 hours and weighed.

### Example 4

2 grams of dried birch (Betula ssp.) sawdust, 10 grams of gamma-valerolactone, 10 grams of 1-pentanol and phosphoric acid were heated in an autoclave at a temperature of 230° C. during 16 minutes. The pressure reached 12.7 bar (absolute). The percentage of residue was determined as described above.

### Example 5

2 gram of dried bagasse (20 mesh particles), 20 grams of levulinic acid and sulfuric acid were loaded in a 30 ml glass flask. The flask was placed in a silicon oil bath and heated to 190° C. After 1 hour, the flask was cooled with water to end the experiment. The percentage of residue was determined by vacuum-filtering the content of the flask, followed by washing the residue with 150 ml of acetone/water (9:1 v/v). The resulting residue was dried at 105° C. for 8 hours and weighed.

In Table 1 is shown the acid concentration (based on the weight of solvent and acid), temperature, heating time and percentage of residue for each of the EXAMPLES 1 to 5.

TABLE 1
Liquefaction of biomass

| | pressure | | | acid conc. | T | | | residue |
|---|---|---|---|---|---|---|---|---|
| EXAMPLE | (bar a) | solvent | acid | (% wt) | (° C.) | time | | (% wt) |
| 1 | 1.0 | γVL | H₃PO₄ | 3 | 200 | 1 | h | 16 |
| 2 | 1.0 | γVL | H₃PO₄ | 7 | 180 | 4 | h | 10 |
| 3 | 1.0 | γVL | H₃PO₄ | 3 | 184 | 3 | h | 18 |
| 4 | 12.7 | γVL/1-pentanol | H₃PO₄ | 3 | 230 | 16 | min | 3 |
| | | (50/50) | | | | | | |
| 5 | 1.0 | levulinic acid | H₂SO₄ | 5 | 190 | 1 | h | 4 |

1. A process for the liquefaction of lignocellulosic or cellulosic material, wherein solid lignocellulosic or cellulosic material is heated at a temperature in the range of from 100 to 300° C. in the presence of an acid catalyst and a solvent, wherein the solvent-to-solid material weight ratio is at most 50, the acid catalyst is present in a concentration of at most 50% by weight of acid based on the weight of solvent and acid, and the solvent comprises a compound having a gamma lactone group of the general molecular formula 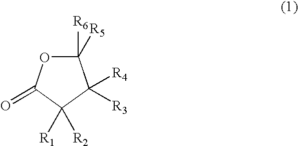
wherein R₁ to R₆ each represent, independently, a hydrogen atom or an organic group connected with a carbon atom to the lactone group.

2. A process according to claim 1, wherein R₁, R₂, R₃ and R₄ each are a hydrogen atom.

3. A process according to claim 2, wherein R₅ is a methyl group, the compound having a gamma lactone group is gamma valerolactone (R6 is a hydrogen atom), 2-methyl-5-oxotetrahydrofuran-2-carboxylic acid (R6 is a carboxyl group), a compound having a molecular structure according to any one of molecular formulas (2) to (5): 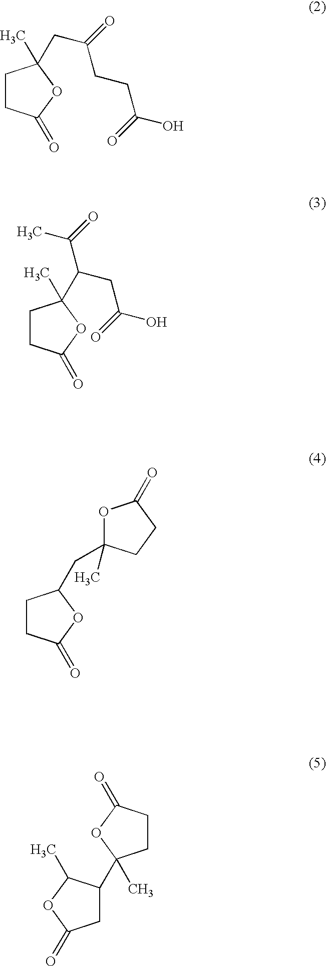
or an ester of a compound having a molecular structure according to molecular formula (2) or (3).

4. A process according to claim 1, wherein the compound having a gamma lactone group has a molecular structure according to molecular formula (6) or (7): 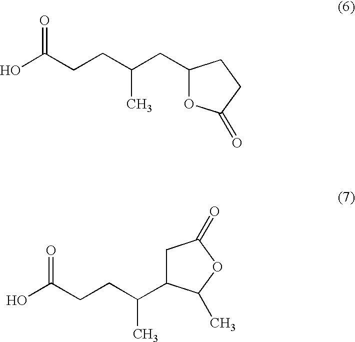
or is an ester of a compound having a molecular structure according to molecular formula (6) or (7).

5. A process according to claim 1, wherein the compound having a gamma lactone group is obtainable from levulinic acid by hydrogenation, dehydration, aldolcondensation, dimerisation or oligomerisation, esterification with an alcohol, or a combination of two or more of these reactions.

6. A process for the liquefaction of lignocellulosic or cellulosic material, wherein solid lignocellulosic or cellulosic material is heated at a temperature in the range of from 100 to 300° C. in the presence of an acid catalyst and a solvent, wherein the solvent-to-solid material weight ratio is at most 50, the acid catalyst is present in a concentration of at most 50% by weight of acid based on the weight of solvent and acid, and the solvent comprises furfural, levulinic acid or a compound obtainable from furfural or levulinic acid by hydrogenation, dehydration, aldolcondensation, dimerisation or oligomerisation, esterification with an alcohol, or a combination of two or more of these reactions.

7. A process according to claim 6, wherein the solvent comprises furfural or a compound obtainable from furfural selected from tetra hydrofurfuryl alcohol, furfuryl alcohol, di-ether of furfuryl alcohol, dimers or oligomers of furfural or furfuryl alcohol.

8. A process according to claim 6, wherein the solvent comprises levulinic acid, an alkyl ester of levulinic acid, a C₅ compound obtainable by hydrogenation and/or dehydration of levulinic acid, or a dimer or oligomer of such C₅ compound, a dimer of levulinic acid obtainable by aldolcondensation, dehydration and optionally hydrogenation, or an alkyl ester of such dimer.

9. A process according to claim 8, wherein the solvent comprises levulinic acid, alpha-angelica lactone, 1,4-pentanediol, 1-pentanol, 4-methyl-6-oxononanedioic acid, the di-methyl or di-ethyl ester of 4-methyl-6-oxononanedioic acid, ethyl levulinate, butyl levulinate, pentyl levulinate, or a combination of two or more thereof.

10. A process according to claim 1, wherein the solvent consists essentially of one or more of the solvent compounds defined in claim 1.

11. A process according to claim 1, wherein the acid catalyst is a strong mineral or organic acid having a pKa below 4.7.

12. A process according to claim 11, wherein the acid is phosphoric acid or sulphuric acid.

13. A process according to claim 11, wherein the acid is oxalic acid, 2-oxopropanoic acid, maleic acid, (1E)-prop-1-ene-1,2,3-tricarboxylic acid, 2,3-dihydroxysuccinic acid, furan-2,5-dicarboxylic acid, or a combination of two or more thereof.

14. A process according to claim 1, wherein the acid catalyst is present in a concentration of at most 20% by weight.

15. A process according to claim 1, wherein the solvent-to-solid material weight ratio is in the range of from 3 to 20.

16. A process according to claim 1, wherein the temperature is in the range of from 120 to 250° C.

17. A process according to claim 1, wherein the pressure is in the range of from 0.1 to 15 bar (absolute).

18. A process according to claim 2, wherein the compound having a gamma lactone group is obtainable from levulinic acid by hydrogenation, dehydration, aldolcondensation, dimerisation or oligomerisation, esterification with an alcohol, or a combination of two or more of these reactions.

19. A process according to claim 3, wherein the compound having a gamma lactone group is obtainable from levulinic acid by hydrogenation, dehydration, aldolcondensation, dimerisation or oligomerisation, esterification with an alcohol, or a combination of two or more of these reactions.

20. A process according to claim 4, wherein the compound having a gamma lactone group is obtainable from levulinic acid by hydrogenation, dehydration, aldolcondensation, dimerisation or oligomerisation, esterification with an alcohol, or a combination of two or more of these reactions.
